(19) United States
(12) Patent Application Publication
(10) Pub. No.: US 2003/0152631 A1
(43) Pub. Date: Aug. 14, 2003

(54) Aseptics

(75) Inventors: Kenji Morishima, Osaka (JP); Norihisa Hatano, Osaka (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: SANTEN PHARMACEUTICAL CO., LTD., OSAKA (JP)

(21) Appl. No.: 10/311,444

(22) PCT Filed: Jun. 13, 2001
(86) PCT No.: PCT/JP01/05004

(30) Foreign Application Priority Data
Jun. 19, 2000 (JP) PAT. 2000-182624

(51) Int. Cl. A61K 31/717; A61K 9/14; A61K 33/22

(52) U.S. Cl. 424/486; 424/658; 424/659; 514/57

(57) ABSTRACT

An object of the present invention is to provide safe preservatives by combining components widely used as additives of aqueous liquids. Preferred preservatives are obtained by combining boric acid and/or borax, ethylenediaminetetraacetic acid or a salt thereof and polyvinyl pyrrolidone. Preservation effects can be enhanced by further combining cellulosic polymers with the preservatives.

### TECHNICAL FIELD

The present invention relates to preservatives comprising a combination of boric acid, ethylenediaminetetraacetic acid and polyvinyl pyrrolidone, and preservatives further containing cellulosic polymers combined therewith. These preservatives can be suitably used for aqueous liquid preparations such as ophthalmic solutions and solutions for contact lenses.

### BACKGROUND ART

Benzalkonium chloride, benzethonium chloride, sorbic acid and the like have been used as preservatives for ophthalmic solutions and solutions for contact lenses.

Though benzalkonium chloride and benzethonium chloride have excellent preservation effects, they are likely to cause corneal disorders depending on concentrations, and the concentrations to be used are limited. Further, these compounds are apt to be adsorbed to contact lenses and plastic containers.

Though sorbic acid, which is widely used as a preservative for ophthalmic solution for contact lenses, exhibits few side-effects and is hardly adsorbed to contact lenses and plastic containers, it has the problem that the preservation effect is weak.

On the other hand, components of the preservatives which can be used for pharmaceuticals such as ophthalmic solutions are limited.

### DISCLOSURE OF THE INVENTION

The present inventors studied precisely to find preservatives which exhibit preservation effects by combining components already used for aqueous preparations such as ophthalmic solutions with each other. Boric acid and/or borax is widely used as a buffer, and ethylenediaminetetraacetic acid or a salt thereof is used as a stabilizer in ophthalmic solutions. Focusing attention on the fact that these compounds have preservation effects, although weak, the present inventors studied to improve the preservation effects. As a result, the present inventors found that the preservation effect is remarkably increased by adding (C) polyvinyl pyrrolidone, which is widely used as a thickener, to (A) boric acid and/or borax and (B) ethylenediaminetetraacetic acid and completed the present invention. It was also found that the preservation effect is further enhanced by adding cellulosic polymers, which are also widely used as thickeners, to the combination.

The preservatives of the present invention consist of three essential components. The first component is boric acid and/or borax. An amount of boric acid and/or borax is preferably 0.05 to 3.0% by weight, more preferably 0.5 to 2.0% by weight. When the amount of the first component is too small, a sufficient preservation effect cannot be obtained. A too large amount is not preferable in terms of safety for eyes.

The second component of the present invention is ethylenediaminetetraacetic acid or a salt thereof. A tetrasodium salt or a disodium salt (disodium edetate) can be suitably used as the salt. An amount of ethylenediaminetetraacetic acid or the salt thereof is preferably 0.01 to 0.3% by weight, more preferably 0.05 to 0.2% by weight. When the amount of the second component is too small, sufficient stability and preservation effect cannot be obtained. A too large amount is not preferable in terms of safety for eyes.

The third component of the present invention is polyvinyl pyrrolidone (PVP). “PVP K-25” (average molecular weight: 25,000), “PVP K-30” (average molecular weight: 40,000) and “PVP K-90” (average molecular weight: 360,000), for example, can be used. An amount of PVP is preferably 0.02 to 4.0% by weight, more preferably 0.1 to 2.0% by weight. When the amount of the third component is too small, a sufficient preservation effect cannot be obtained. A too large amount is not preferable since unpleasant stickiness is felt.

The preservation effect is further enhanced by adding the cellulosic polymer to the above-mentioned three components of the present invention. Examples of the cellulosic polymer are hydroxypropylmethyl cellulose, hydroxypropyl cellulose, methyl cellulose and hydroxyethyl cellulose. Hydroxypropylmethyl cellulose is particularly preferable. An amount of the cellulosic polymer is preferably 0.01 to 0.5% by weight, more preferably 0.05 to 0.3% by weight, but not limited to the ranges.

Since the preservatives of the present invention are safe for the human body and hardly adsorbed to contact lenses and plastic containers, these are suitably used for aqueous liquid preparations such as ophthalmic solutions and solutions for contact lenses. It is possible to add appropriately further additive components such as an isotonic agent, a pH adjustor, a solubilizer and another preservative to the above-mentioned components in order to prepare these aqueous liquid preparations.

Drugs to which these aqueous liquid preparations can be applied are not particularly limited and are exemplified by various vitamins (vitamin B2, vitamin B6, vitamin B12, vitamin E, panthenol and the like), decongestants (tetrahydrozoline hydrochloride, naphazoline hydrochloride and the like), anti-inflammatories (disodium glycyrrhizinate, ε-aminocapronic acid and the like), antihistamines (chlorpheniramine maleate, diphenhydramine hydrochloride and the like), antiallergics (sodium cromoglicate and the like), antimicrobials (sulfamethoxazole and the like), amino acids (potassium L-aspartate, aminoethylsulfonic acid, sodium chondroitin sulfate and the like), sodium hyaluronate, neostigmine methylsulfate and the like.

Examples of the isotonic agent are glycerin, propylene glycol, sodium chloride, potassium chloride, sorbitol and mannitol.

Examples of the pH adjustor are hydrochloric acid, citric acid, phosphoric acid, acetic acid, sodium hydroxide, potassium hydroxide, sodium carbonate and sodium hydrogencarbonate.

Examples of the solubilizer are Polysorbate 80, polyoxyethylene hydrogenated castor oil 60 and macrogol 4000.

The preservative of the present invention can be combined with a widely-used preservative such as sorbic acid, potassium sorbate, benzalkonium chloride, benzethonium chloride, methyl p-hydroxybenzoate, propyl p-hydroxybenzoate and chlorobutanol. Since the preservative of the present invention can complement a preservation effect of the widely-used preservative owing to the combination, the preservative of the present invention has also an advantageous effect on decreasing an amount of the widely-used preservative remarkably.

When the-liquid preparations of the present invention are used as ophthalmic solutions, it is desirable to adjust pH to about 7.0, and it is desirable to adjust an osmotic pressure ratio to about 1.0.

The present invention is described in detail by giving Examples below, but these Examples do not limit the scope of the present invention.

### BEST MODE FOR CARRYING OUT THE INVENTION

Preservation effect tests were carried out according to the following method in order to study preservation effects of the preservative of the present invention.

### Preservation Effect Tests

In Examples 1 to 4 and Comparative Examples 1 to 3, formulation ingredients shown in Table 1 were added to distilled water by the conventional method to prepare liquid preparations. Sodium chloride as an isotonic agent was added to each liquid preparation to adjust osmotic pressure to 1.0, and further sodium hydroxide was optionally added to the liquid preparation to adjust pH to 7.0. Preservation effect tests were carried out according to the preservation effect test method of 13th revised Japanese Pharmacopoeia. Staphyrococcus aureus (S.aureus) was used as test bacteria, and survival rates of the bacteria were calculated according to the following equation. The obtained values are shown in Table 1. Survival rate (%)=[(Bacteria number after two weeks)/(Initial bacteria number)]×100

TABLE 1

| Formulation | | | | | Comparative | | |
|---|---|---|---|---|---|---|---|
| ingredient | Examples | | | | Examples | | |
| (% by weight) | 1 | 2 | 3 | 4 | 1 | 2 | 3 |
| Boric acid | 1.5% | 1.39% | 1.5% | 1.39% | 1.5% | — | — |
| Borax | — | 0.18% | — | 0.18% | — | — | — |
| Na edetate | 0.1% | 0.1% | 0.1% | 0.1% | 0.1% | | |
| PVP*¹ | 1.0% | 0.2% | 1.0% | 1.0% | — | 1.0% | — |
| HPMC*² | — | — | 0.2% | 0.3% | — | — | 0.1% |
| Survival rate (%) | 0.50 | 0.59 | 0.19 | 0.03 | 1.7 | 6.2 | 7.9 |

*¹PVP: Polyvinyl pyrrolidone
*²HPMC: Hydroxypropylmethyl cellulose

Table 1 explicitly shows that preservation effects are improved remarkably in Examples 1 and 2 of the present invention compared with those in Comparative Examples 1 to 3. Preservation effects are improved in Examples 3 and 4 wherein HPMC is further added compared with those in Examples 1 and 2. Thus, the preservation effects of the preservatives of the present invention containing (A) boric acid and/or borax, (B) ethylenediaminetetraacetic acid or a salt thereof and further (C) polyvinyl pyrrolidone are improved by a synergistic action of these components. The preservation effects of the present invention can be more improved by further adding the cellulosic polymer to the preservatives. Since the preservatives of the present invention are prepared to improve the preservation effects of the liquid preparations by combining the compounds widely used as a buffer, a stabilizer and a thickener respectively, the preservatives are safe for the human body and appropriate to pharmaceutical use. The preservatives of the present invention can also be combined with a widely-used preservative such as benzalkonium chloride or sorbic acid, and an amount of the widely-used preservative can be reduced owing to the combination.

Industrial Applicability

The present invention provides preservatives comprising a combination of boric acid, ethylenediaminetetraacetic acid and polyvinyl pyrrolidone, and preservatives further containing cellulosic polymers combined therewith. The preservatives can be suitably used for aqueous liquid preparations such as ophthalmic solutions and solutions for contact lenses.

1. A preservative comprising a combination of (A) boric acid and/or borax, (B) ethylenediaminetetraacetic acid or a salt thereof and (C) polyvinyl pyrrolidone.

2. The preservative as claimed in claim 1, characterized in that (D) a cellulosic polymer is further combined therewith.

3. An aqueous liquid preparation containing the preservative as claimed in claim 1 or 2.

4. The aqueous liquid preparation as claimed in claim 3, wherein a dosage form is an ophthalmic solution.

5. An aqueous liquid preparation comprising a combination of (A) boric acid and/or borax in an amount of 0.05 to 3.0% by weight, (B) ethylenediaminetetraacetic acid or a salt thereof in an amount of 0.01 to 0.3% by weight and (C) polyvinyl pyrrolidone in an amount of 0.02 to 4.0% by weight as a preservative.

6. The aqueous liquid as claimed in claim 5, which further comprises (D) a cellulosic polymer in an amount of 0.01 to 0.5% by weight.

7. The preservative as claimed in claim 2 or the aqueous liquid as claimed in claim 6, wherein the cellulosic polymer is hydroxypropylmethyl cellulose, hydroxypropyl cellulose, methyl cellulose or hydroxyethyl cellulose.
